(19) United States
(12) Patent Application Publication
(10) Pub. No.: US 2014/0045801 A1
(43) Pub. Date: Feb. 13, 2014

(54) PRAMIPEXOLE TRANSDERMAL DELIVERY FOR SEVERE HEADACHES

(71) Applicant: David Thomas Rossi, Morgantown, WV (US)

(72) Inventor: David Thomas Rossi, Morgantown, WV (US)

(73) Assignee: MYLAN INC., Morgantown, WV (US)

(21) Appl. No.: 13/570,593

(22) Filed: Aug. 9, 2012

(51) Int. Cl. A61K 31/428; A61K 31/4439; A61K 31/4418; A61K 31/485; A61K 31/573; A61P 25/06; A61K 31/48

(52) U.S. Cl. 514/171; 514/367; 514/343; 514/250; 514/282; 514/356

(57) ABSTRACT

The present invention relates to a method for the treatment and prevention of cluster headaches and migraines using the transdermal administration of pramipexole.

### BACKGROUND OF THE INVENTION

Cluster headaches are those headaches which recur over a period of time. Patients inflicted with a cluster headache may experience an episode one to three times a day during the cluster period which may range from two weeks to three months. Migraines are chronic neurological disorders causing severe headaches and nausea. Typical migraines affect one half of the head and last from about 2 to about 72 hours. Common initial treatment for cluster headaches and migraines consist of the administration of oral pharmaceutical dosage forms.

Pramipexole is a non-ergoline dopamine agonist indicated for treating early-stage Parkinson's disease as well as restless legs syndrome. Pramipexole dihydrochloride is widely prescribed and used as daily doses of solid-oral tablets available in strengths of 0.125, 0.25, 0.5, 0.75, 1.0, and 1.5 mg.

Transdermal drug delivery routes are often preferred over other types of medication delivery, such as oral or topical, due to the controlled release of the medication into the patient over several hours or days. Transdermal delivery systems, however, can only be employed with molecules which are small enough and of the correct lipophilicity to penetrate the skin, as the skin acts as a very effective barrier.

Pramipexole free base is a small molecule with moderate lipophilicity (log P of 2.35), and therefore is suitable for transdermal delivery.

Transdermal pramapexole patches are fairly common as they are used in the treatment of Parkinson's disease as well as other disease states including schizophrenia, restless leg syndrome, tardive dyskinesia and movement disorders, addictive disorders, sleep disorders, neurological impairment associated with brain injury, depression, HIV dementia, other dementias, obesity, diabetes, anhedonia, attention deficit-hyperactivity disorder, tremors, enhancement of female desire, retinal tissue restoration, neuro-inflammatory disorders, smoking cessation neurodegenerative diseases and neuropathic pain.

The use of the transdermal delivery of pramipexole for, the treatment and prevention of reoccurring migraine and cluster headaches was previously unknown.

### SUMMARY OF THE INVENTION

The invention provides for a method for the treatment or prevention of severe headaches, including migraine headaches, cluster headaches, reoccurring cluster headaches, chronic cluster headaches, chronic cluster headaches unremitting from the offset and the like, using a transdermal patch comprising pramipexole. Such patch may include additional concomitant medicine.

### DETAILED DESCRIPTION OF THE INVENTION

It has now been found that Pramipexole transdermal patches may be used for the treatment and prevention of cluster headaches and migraines. The transdermal administration of the drug is superior over the oral delivery for the intended uses as the drug can be given in a slow, controlled release manner and can be administered once to last for 1 to 7 days. Further, transdermal delivery will be useful for delivering lower doses of the drug which can decrease the occurrence of the side-effects of pramipexole including orthostatic hypotension. Additionally, a slow, controlled release of transdermal pramapexole will have the greatest benefit for those who have experienced their first migraine or cluster headache and are expecting to have reoccurring headaches.

The structure/type of transdermal patch is not critical so long as its components are compatible with the pramipexole compound as well as any additional active pharmaceutical compounds that may be present. With this proviso in mind, any type of transdermal patch may be used, including, but not limited to, a single layer drug in adhesive, multi-layer drug in adhesive, reservoir patch, monolithic patch, vapor patch or other form of transdermal patches. This invention, while relying in part on known forms of transdermal patch delivery, is generally independent of the structural aspects of the patch, in that all transdermal patches can efficiently deliver the drug at the intended amounts, rates and durations.

Using conventional transdermal patch technology known in the art of pharmaceutical sciences, Pramipexole free base can be delivered in doses of from 0.01 to 10 mg per day. The duration of drug delivery from a transdermal device may last from 1 to 7 days. The rate of administration of drug to the patient will be in the range from 1 μg to 200 μg per hour.

It is well known in the art of pharmaceutical sciences that non-ionized drugs, such as free acids and free bases, most readily lend themselves to transdermal delivery. Although it may be possible to transdermally deliver a pramapexole salt, such as the dihydrochloride salt or a more lipophilic salt such as the besylate salt, the preferred chemical form for transdermal delivery will be free-base pramapexole. The non-ionized free base will be more likely to pass through the layers of skin to reach the underlying blood vessels and therefore be bioavailable to the patient.

The transdermal use of Pramipexole is found to be useful for the treatment of cluster and migraine headaches, and the prevention of cluster headaches, migraine headaches, reoccurring migraine headaches, reoccurring cluster headaches, chronic cluster headaches, and chronic migraine headaches.

The Pramipexole may be delivered transdermally by itself or in combination with one or more other drug(s) useful for treating or preventing severe headaches. Such concomitant medications include, but are not limited to serotonin receptor agonists, NSAIDS, antidepressants, ergotalkaloids, opiods, antiepileptics, anti-seizure drugs, calcium channel blockers, and beta blockers. Such other drug(s) may be contained within the transdermal patch or administered separately.

Examples of serotonin receptor agonists include, but are not limited to, sumatriptan, elitriptan, frovotriptan, zolmitriptan, naratriptan, rizatriptan, and almotriptan.

Examples of NSAIDS include, but are not limited to, ibuprofen, naproxen, aspirin, acetaminophen, indomethacin, and ketoprofen.

An example of an antidepressant includes, but is not limited to, amitriptyline.

An example of an ergotalkaloid includes, but is not limited to, ergotamine.

Examples of opiods include, but are not limited to, oxycodone and hydrocodone.

Examples of antiepileptics include, but are not limited to, gabapentin, topiramate, and levetiracetam.

An example of an anti-seizure drug includes, but is not limited to, sodium valerate.

Examples of calcium channel blockers include, but are not limited to, verapamil and amlodipine.

Examples of beta blockers include, but are not limited to, propanolol, metoprolol, cavedilol, and atenolol.

1. A method of treating and preventing cluster headaches and migraines in a human patient, comprising administering pramipexole transdermally to said human patient.

2. The method of claim 1, wherein the transdermal delivery is in the form of a patch.

3. The method of claim 2, wherein the transdermal patch is a form selected from the group consisting of a single layer drug in adhesive, a multi-layer drug in adhesive, a reservoir patch, a monolithic patch, and a vapor patch.

4. The method of claim 1 wherein the cluster headaches and migraines are reoccurring or chronic.

5. The method of any one of claims 1-4, wherein the pramipexole is delivered in doses of from 0.01 mg to 10 mg per day for 1 to 7 days.

6. The method of claim 5, wherein the rate of administration of drug to patient ranges from 1 μg to 200 μg per hour.

7. The method of any one of claims 1-6, wherein the pramipexole is delivered in its free base form.

8. The method of any one of claims 1-7, further comprising an additional drug useful for treating or preventing headaches.

9. The method of claim 8, wherein the additional drug is selected from the group consisting of a serotonin receptor agonist, an NSAID, an antidepressant, an ergotalkaloid, an opioid, an antiepileptic, an anti-seizure drug, a calcium channel blacker, and a beta blocker.

10. The method of claim 9, wherein the serotonin receptor agonist is selected from the group consisting of sumatriptan, elitriptan, frovotriptan, zolmitriptan, naratriptan, rizatriptan, or almotriptan.

11. The method of claim 9, wherein the NSAID is selected from the group consisting of ibuprofen, naproxen, aspirin, acetaminophen, indomethacin, and ketoprofin.

12. The method of claim 9, wherein the antidepressant is amitriptyline.

13. The method of claim 9, wherein the ergotalkaloid is ergotamine.

14. The method of claim 9, wherein the opioid is selected from the group consisting of oxycodone and hydrocodone.

15. The method of claim 9, wherein the antiepileptic is selected from the group consisting of gabapentin, topiramate, and levetiracetam.

16. The method of claim 9, wherein the anti-seizure drug sodium valerate.

17. The method of claim 9, wherein the calcium channel blocker is selected from the group consisting of verapamil and amlodipine.

18. The method of claim 9, wherein the beta blocker is selected from the group consisting of propanolol, metoprolol, carvedilol, and atenolol.
